(19) United States
(12) Patent Application Publication
(10) Pub. No.: US 2015/0282784 A1
(43) Pub. Date: Oct. 8, 2015

(54) Ultrasound (US) Imaging Probe With Rotating Transducer Array

(71) Applicant: Analogic Corporation, Peabody, MA (US)

(72) Inventor: Guy M. Besson, Peabody, MA (US)

(21) Appl. No.: 14/244,262

(22) Filed: Apr. 3, 2014

(51) Int. Cl. A61B 8/00; A61B 8/08

(52) U.S. Cl. CPC A61B 8/4461; A61B 8/5207

(57) ABSTRACT

An ultrasound imaging probe includes a housing having a tubular portion with a first long axis and a first end region having a first non-zero diameter, a non-zero height, and an inner circular perimeter that surrounds a material free region. The probe further includes a transducer array support disposed in the material free region and mechanically supported by the housing. The probe further includes a transducer array with a row of transducer elements with a transducing side and a second long axis. The transducer array is disposed in the material free region such that the second long axis extends within the perimeter and is perpendicular to the first long axis, and is rotatably affixed to the transducer array support on the first long axis with the transducing side facing out of the housing and the transducer array configured to rotate in a plane parallel to the circular inner perimeter.

### TECHNICAL FIELD

The following generally relates to ultrasound (US) imaging and more particularly to volumetric ultrasound imaging with a rotating transducer array.

### BACKGROUND

Ultrasound (US) imaging provides useful information about the interior characteristics (e.g., anatomical tissue, material flow, etc.) of a subject under examination. An ultrasound imaging system has included a probe with an ultrasound transducer array and a console with controls for controlling the transducer elements of the transducer array to transmit an ultrasonic beam and receive echoes produced in response thereto. The console has also included a processor that processes the echoes and a rendering engine which visually displays the processed echoes.

A 1D transducer array includes a single row of transducer elements. A width of the elements is on the order of a wave length. By controlling the delays and weight coefficients in the beamforming, the focus can be controllably moved along a line. In the elevation direction, the height has been several millimeters. The focusing in the elevation plane is achieved with acoustic lenses, and the focus is generally fixed. The beam is narrowest at the elevation focus and diverges beyond it. Close to the transducer, the beam is as wide as the transducer array, and away from the elevation focus, the beam is wider.

A 1.5D transducer array has several rows of elements. The effective size of the elements in elevation direction is usually much larger than the width. The outer rows are electrically connected to the middle row. A switch alternately couples outer rows to the middle row, depending on the distance from the transducer surface, creating large elements at large depths. Such arrays have had acoustic lenses that focus the beam in elevation direction. A 1.75D array is similar to a 1.5D array, but each element is connected to a channel. This allows electronic focusing in the elevation direction.

A 2D transducer array includes a matrix of transducer elements, which allows for volumetric imaging. However, relative to its 1D counterpart, a 2D array includes significantly more transducer elements (e.g., N², for a square matrix, compared to N for a single row 1D transducer array), channels and electrical interconnects, increasing complexity and/or the physical footprint, and, unfortunately, tends to be significantly more costly than its 1D counterpart.

### SUMMARY

Aspects of the application address the above matters, and others.

As described herein, a hand-held ultrasound probe includes a rotating transducer for “real-time” volumetric ultrasound imaging, using a sparse array comprising one or several linear arrays; one or several linear arrays comprising a plurality of rows, or one or several arrays of ultrasound transducer cells arranged in an optimized sparse geometry on the surface of a rotating support element. The transducer array rotates about a long axis, which is also the axis of a non-rotatable tubular housing, with the transducing surface of the transducer array facing out of the housing and at the tissues to be imaged.

In one aspect, an ultrasound imaging probe includes a housing having a tubular portion with a first long axis and a first end region having a first non-zero diameter, a non-zero height, and an inner circular perimeter that surrounds a material free region. The probe further comprising a transducer array support disposed in the material free region and mechanically supported by the housing. The probe further comprising a transducer array with a row of transducer elements with a transducing side and a second long axis. The transducer array is disposed in the material free region such that the second long axis extends within the perimeter and is perpendicular to the first long axis. The transducer array is rotatably affixed to the transducer array support on the first long axis with the transducing side facing out of the housing and the transducer array configured to rotate in a plane parallel to the circular inner perimeter.

In another aspect, a method includes receiving, at a user interface of an ultrasound probe, an input signal indicative of an ultrasound volumetric imaging mode. The method further includes rotating, in response to receiving the input signal, a row of transducer elements of a transducer array of an ultrasound imaging probe in an examination region plane about a central region of the a row of transducer elements. The method further includes transmitting ultrasound signals and acquiring echoes with the row of transducer elements while rotating the row of transducer elements. The method further includes processing the echoes, generating volumetric image data. The method further includes visually presenting the generated volumetric image data.

In another aspect, an ultrasound system includes an ultrasound imaging probe with a row of transducer elements, a console that controls the row of transducer element to transmit ultrasound signals and receive echo signals, and means for rotating the transducer array relative to the ultrasound imaging probe to acquire volumetric image data.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 illustrates an example ultrasound imaging system including a hand-held ultrasound probe with a rotating transducer array;

FIG. 2 schematically illustrates a perspective view of an example of the hand-held ultrasound probe;

FIG. 3 schematically illustrates a cross-sectional view along line A-A of the example hand-held ultrasound probe of FIG. 2;

FIG. 4 schematically illustrates a perspective view of another example of the hand-held ultrasound probe;

FIG. 5 schematically illustrates a cross-sectional view along line A-A of another example of the hand-held ultrasound probe of FIG. 2;

FIG. 6 schematically illustrates the hand-held ultrasound probe with a 1D linear array with equal size transducer elements;

FIG. 7 schematically illustrates the hand-held ultrasound probe with a 1D linear array with different size transducer elements;

FIG. 8 schematically illustrates the hand-held ultrasound probe with a plurality of 1D linear arrays or a 2D array;

FIG. 9 schematically illustrates the hand-held ultrasound probe with multiple 1D sparse arrays;

FIG. 10 schematically illustrates the hand-held ultrasound probe with a concentric array;

FIG. 11 schematically illustrates the hand-held ultrasound probe with a 1.5D or 1.75D array;

FIG. 12 illustrates an example of the ultrasound imaging system;

FIG. 13 schematically illustrates a variation of hand-held ultrasound probe; and

FIG. 14 illustrates a method in accordance with the embodiments disclosed herein.

### DETAILED DESCRIPTION

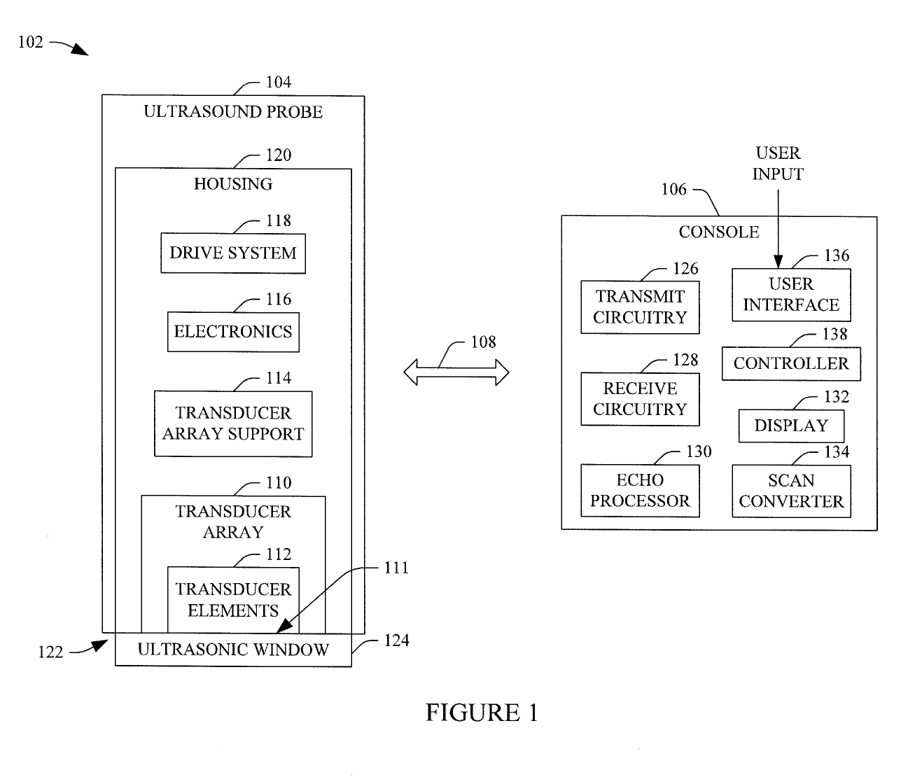

FIG. 1 schematically illustrates an example imaging system 102, such as ultrasound (US) imaging system. The imaging system 102 includes a hand-held ultrasound probe 104 and a console 106. The hand-held ultrasound probe 104 and the console 106, in the illustrated embodiment, are in electrical communication through an interface 108, such as a wireless (e.g., RF, IF, optical, etc.) and/or a physical (e.g., electro-mechanical) interface. The hand-held ultrasound probe 104 and the console 106 can be part of a same device and/or separate and distinct devices.

The hand-held ultrasound probe 104 includes a transducer array 110 such as a 1D, 1.5D, 1.75D, 2D, and/or other transducer array. A multi-row 1D configuration and/or the 1.5D, 1.75D, and 2D configurations include one or more additions rows, but, generally, in the illustrated embodiment, not enough rows to cover the entire 2D field of view when the transducer array 110 is maintained at a static position relative to the probe 104. The transducer array 110 includes a plurality of transducer elements 112 in each row of elements. For example, a row of transducer elements may include 8, 64, 128, 256, 512, etc. transducer elements 112.

A row transducer elements of the transducer array 110 can be linear, curved, and/or otherwise shaped. At least two different rows transducer elements of the transducer array 110 may be differently shaped (e.g., one row may be linear and another row may be curved). An individual row transducer elements of the transducer array 110 can be fully populated or sparse. With a multi-row transducer array 110, at least two rows of transducer elements can be arranged with respect to each other parallel, perpendicular, or at another angle, such as an angle there between. At least two transducer elements 112 of the transducer array 110 may also have different geometry (e.g., width or height).

The transducer elements 112 have a transducing side 111, which faces away from the probe 104 and into the field of view. A transducer element 112 of the transducer elements 112 converts an electrical signal received thereby into an ultrasound pressured field and vice versa. For example, the transducer elements 112 of a row, when excited with an electrical pulse(s), transmit ultrasound signals from the transducing side 111 into a field of view and receive, at the transducing side 111, echo signals generated in response to an interaction of the transmitted ultrasound signals with structure in the field of view, which are converted into electrical signals indicative of the structure.

The hand-held ultrasound probe 104 further includes a transducer array support 114, which mechanically supports the transducer array 110 and hence the transducer elements 112 in the ultrasound probe 104. The transducer array support 114 rotatably supports transducer array 110. The hand-held ultrasound probe 104 further includes electronics 116 and a drive system 118. The electronics 116 include electrically conductive paths between the plurality of transducer elements 112 and the interface 108. The drive system 118 (e.g., a motor and a shaft coupling, a belt, a chain, a gear, etc.) rotates the transducer array 110.

As described in greater detail below, by rotating the transducer array 110 at a suitable speed while acquiring data, the transducer array 110 can be used to acquire volumetric data with one to a few rows of transducer elements 112. Thus, the hand-held ultrasound probe 104 described herein can acquire, via rotating the transducer array 110, volumetric data of a field of view with less transducer elements 112 relative to a fully populated 2D configuration of the transducer array 110 that covers the entire field of view. As such, the hand-held ultrasound probe 104 described herein includes less channels and electrical interconnects, and is less complex and costly and smaller, relative to such a 2D configuration.

The hand-held ultrasound probe 104 further includes a housing 120. The housing 120 includes structural elements, components, etc., which physically and/or mechanically support the transducer array 110, the transducer array support 114, the electronics 116 and/or the drive system 118. Where the console 106 is part of the hand-held ultrasound probe 104, the display 132 and/or user interface 136 may be part of and/or integrated with the housing 120, for example, the display 132 and/or user interface 136 can be a physical part of a side of the hand-held ultrasound probe 104.

The hand-held ultrasound probe 104 further includes an ultrasonic window 124. The ultrasonic window 124 resides between the transducer elements 112 and the environment outside of the housing 120. Similar to the display 132 and/or user interface 136, the ultrasonic window 124 may be part of and/or integrated with the housing 120. The ultrasonic window 124 allows signals transmitted by the transducer array 100 to exit the housing 120 and enter the field of view, and echoes from the field of view enter the housing 120 to and are received by the transducer array 100.

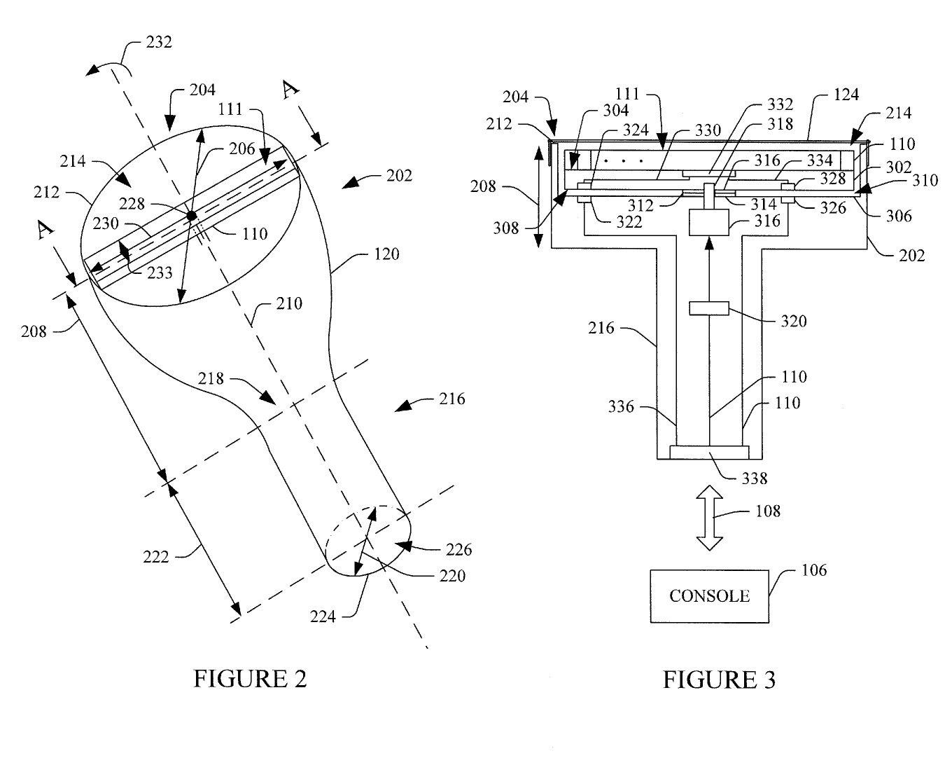

In one instance, the housing 120 can be grasped by a mechanical or robotic arm, a human hand, and/or otherwise and maneuvered by the mechanical or robotic arm, the human hand, etc. for an ultrasound procedure. Non-limiting examples of different configurations are described in connection with FIGS. 2, 3, 4 and 5. In another instance, the hand-held ultrasound probe 104 can be part of another physical structure, such as an instrument. An example of a suitable instrument is a catheter, a lumen, and/or other instrument. An example of this configuration is described in connection with FIG. 13.

The console 106 includes transmit circuitry 126 that conveys a set of pulses that selectively excites one or more of the transducer elements 112 to transmit an ultrasound signal into a scan field of view.

The console 106 further includes receive circuitry 128 that receives a set of echoes signals. The echo signals, generally, are a result of the interaction between the emitted ultrasound signals and the object (e.g., flowing blood cells, organ cells, etc.) in the scan field of view.

The console 106 further includes an echo processor 130 that processes the received echoes, e.g., by applying time delays and weights to the echoes and summing the resulting echoes. Other processing by the echo processor 130 and/or other component includes, but is not limited to, spatial compounding, filtering (e.g., FIR and/or IIR), and/or other echo processing.

The console 106 further includes a display 132 that visually presents images and/or other information. The console 106 further includes a scan converter 134 that scan converts the processed data, e.g., by converting the beamformed data to the coordinate system of the display 132.

The console 106 further includes a user interface (UI) 136 with one or more input devices (e.g., a button, a knob, a slider, etc.), which allow interaction between with the system 102 and a user.

The console 106 further includes a controller 138 that controls the various components of the imaging system 102. For example, such control may include exciting individual or groups of the transducer elements 112, steering and/or focusing the transmitted signal, etc., steering and/or focusing the received echoes, invoking the electronics 116 and drive system 118 to rotate the transducer array 110, etc.

It is to be appreciated that the console 106 includes one or more processors (e.g., a microprocessor, a central processing unit, etc.) that execute one or more computer readable instructions encoded or embedded on computer readable storage medium (which excludes transitory medium) such as physical memory and other non-transitory medium. Additional or alternatively, the instructions can be carried in a signal, carrier wave and other transitory or non-computer readable storage medium. By executing the instructions, the one or more processors implement one or more of the components 126, 128, 130 and/or 134.

The hand-held ultrasound probe 104 and the console 106 may be separate devices. In this configuration, the hand-held ultrasound probe 104 and the console 106 have interfaces for communication between each other, over a hard wired and/or wireless channel. For example, the hand-held ultrasound probe 104 and the console 106 may each include electro-mechanical connectors. With this configuration, a cable or like includes complementary connectors in that the cable connectors physically engage the hand-held ultrasound probe 104 and the console 106 connectors and provides an electrical pathway between the hand-held ultrasound probe 104 and the console 106. Such a cable could be part of one of the hand-held ultrasound probe 104 and the console 106 which removably connects to the other of the hand-held ultrasound probe 104 and the console 106.

In another instance, the console 106 is part of and/or integrated within the hand-held ultrasound probe 104. In this configuration, the hand-held ultrasound probe 104 may include internally located power, e.g., from a power source such as a battery, a capacitor or other power storage device located in the housing 102, to power the components therein. The hand-held ultrasound probe 104 may additionally or alternatively use external power. In another instance, at least one of the transmit circuitry 126, the receive circuitry 128, the echo processor 130, the scan converter 134, the controller 138, the display 130 or the user interface 136 is separate from the probe 104.

Optionally, the ultrasound imaging system 102 may include a location and guidance elements to determine in three-dimensional space the rotation axis direction, the contact surface location, and the instantaneous rotating probe position.

Turning to FIG. 2, an example perspective view of the hand-held ultrasound probe 104 is schematically illustrated.

The housing 120 includes a tubular portion 202 with a first end region 204 having a first non-zero diameter 206, a non-zero height 208, and a long axis 210, which extends along a center of the tubular portion 202. An example of a suitable diameter is a diameter in a range of four (4) to twenty (20) centimeters (cm), and an example of a suitable height is a height in a range of 1 to 5 cm. The tubular portion 202 includes an inner circular perimeter 212 and a material free region 214 surrounded and/or enclosed by the inner circular perimeter 212. The tubular portion 202 also includes a side 218 opposing the first end region 204.

The housing 120 further includes a handle portion 216, which is affixed to and/or is part of the side 218. In this example, the handle portion 216 is also centered along the axis 210. The illustrated handle portion 216 is also tubular having a non-zero diameter 220 and a non-zero height 222. An example of a suitable diameter is a diameter in a range of one (1) to ten (10) cm, and an example of a suitable height is a height in a range of 7 to 20 cm. The handle portion 216 includes a perimeter 224 and a material free region 226 surrounded and/or enclosed by the perimeter 224. In a variation, the handle portion 216 is not tubular, but instead, otherwise shaped such a square, hexagonal, elliptical, end/or other shape.

The transducer array 110 is rotatably affixed to a structural rotation support 228, which runs along the long axis 210, perpendicular to the long axis 210 and facing away from the housing 120. In the illustrated embodiment, the transducer array 110 is affixed to the support at about a central region of the transducer array 110 or the transducer elements 112. The structural rotation support 228 can be a pin, a rod, a shaft, etc., and the transducer array 110 is configured to rotate (e.g., as shown at 232) three hundred and sixty (360) degrees about the structural rotation support 228. The transducer array 110 has a length 2030 in a range of 3 to 12 cm and a width 233 in a range of 0.1 to 2 cm, and an area (i.e., length×width), which is less than an area of a configuration in which the transducer array 110 was a fully populated circular array with an area of approximately πr², where r is half of the length 230. The transducer array 110 is configured to rotate and acquire data as it rotates so as to acquire data over such an area.

By way of non-limiting example, the speed of sound in water is on the order of 1500 m/s. If the hand-held ultrasound probe 104 were to be used to image an object at a depth of about fifteen (15) cm in a medium with characteristics (e.g., reflecting and/or scattering boundaries) similar to water, the round trip distance would be thirty (30) cm (or 0.30 m), and the transducer array 110 would need to transmit and receive within two microseconds (200 μsec) to image one line with the transducer array 110 before the next acquisition interval.

In general, the transducer array 110 is configured to rotate in a range of 0.10 to several rotations per second. For a particular scan, this number depends on the number of rows, the width of each row, and the probe diameter. For example, with two rows of 1-mm and a diameter of 100 mm, the circumference is about 314 mm. At the periphery, to advance 1 mm in 200 microsecond (or 314 mm in 62.8 ms), the array 110 is rotated no faster than approximately 16 rotations per seconds. Imaging is also possible with a single row, if the row does not advance by more than half its width until the echo comes back.

FIG. 3 shows a cross-sectional view along line A-A of FIG. 2.

The transducer array support 114 is disposed entirely in the material free region 214 and is mechanically supported by the housing 120. The transducer array support 114 includes rotating support 302, which is recessed in the material free region 214 within the perimeter 212 along the height 208. The transducer array 110 is affixed to a side 304 of the rotating support 302 and is also recessed in the perimeter 212 from the first end region 204 along the height 208 disposed entirely in the material free region 214. The transducer array support 114 further includes a stationary support 306, which is located next to a side 308 of the rotating support 302, which is opposite the side 304 of the rotating support 302.

The stationary support 306 and the rotating support 302 are separated by a non-zero gap 310. The gap 310 is in the range of about 0.5 to 2 mm. A bearing 312 resides at least partially in the non-zero gap 310, between the stationary support 306 and the rotating support 302. The bearing 312 includes a stationary bearing 314 and a rotating bearing 316. The stationary bearing 314 is affixed to the stationary support 306, and the rotating bearing 312 is affixed to the rotating support 302. The bearing 312 allows the rotating support 302, and hence the transducer array 110, to rotate relative the stationary support 306. The bearing 312 can be a ball bearing, a roller bearing, etc.

A motor 316 turns a shaft 318, which is fixedly attached to the rotating support 302, which rotates the transducer array 110. In other embodiments, the motor 316 drives a belt, a chain, a gear, and/or other device which directly or indirectly rotates the transducer array 110. A motor controller 320 controls the motor. A probe interface 338 receives a control signal from the console 106 and routes the control signal to the motor controller 320. The control signal, in one instance, indicates a rotational velocity at which to rotate the transducer array 110 for an ultrasound imaging procedure, and the motor controller 320 invokes the motor 316 to rotate the shaft as a rotational velocity which will cause the transducer array to rotate at the commanded rotational velocity.

A pair of electrical contacts 322 and 324 provides an electrical path from the stationary support 306 to the rotating support 302. The pair of electrical contacts 322 and 324 may include physical contacts such as metallic brush contacts or contactless contacts. A pair of transceivers 326 and 328 provides a data and control signal path between the rotating support 302 and the stationary support 306. The pair of transceivers 326 and 328 may include an optical, a radio frequency (RF), an infrared (IR), and/or other transceivers. In a variation, the pair of transceivers 326 and 328 is omitted, and data and control signals are also routed between the stationary support 306 to the rotating support 302 via the pair of electrical contacts 322 and 324 and/or other electrical contacts.

A first electrical path 330 provides an electrical path between the electrical contact 324 and electronics 332 of the transducer array 110. Another different electrical path 334 provides an electrical path between the transceiver 328 and electronics 332 of the transducer array 110. Another different electrical path 336 provides an electrical path between the electrical contact 322 and the interface 338. Another different electrical path 340 provides an electrical path between the transceiver 326 and the interface 338. The interface 338 can includes a wireless interface and/or an electromechanical connector. The location of the pair of electrical contacts 322 and 324, the pair of transceivers 326 and 328, and the electrical paths 330, 334, 336 and 340 are for explanatory purposes and are not limiting; other locations are contemplated herein.

In FIG. 3, the ultrasonic window 124 includes a cover or the like that extends over the material free region 214 and is affixed to the perimeter 212 at the first end region 204. The ultrasonic window 124 resides between the transducer elements 112 and the environment outside of the housing 120. In one non-limiting instance, the ultrasonic window 124 includes a taught membrane including an appropriate material for the transmission of ultrasound beam energy. The taught membrane can be a disc planar shape and/or other shape. The taught membrane, in this instance, minimizes tissue displacement when the transducer array 110 rotates and the ultrasonic window 124 is in contact with the structure being imaged. In a variation, the ultrasonic window 124 is omitted. In another variation, the ultrasonic window 124 can be part of the housing 120.

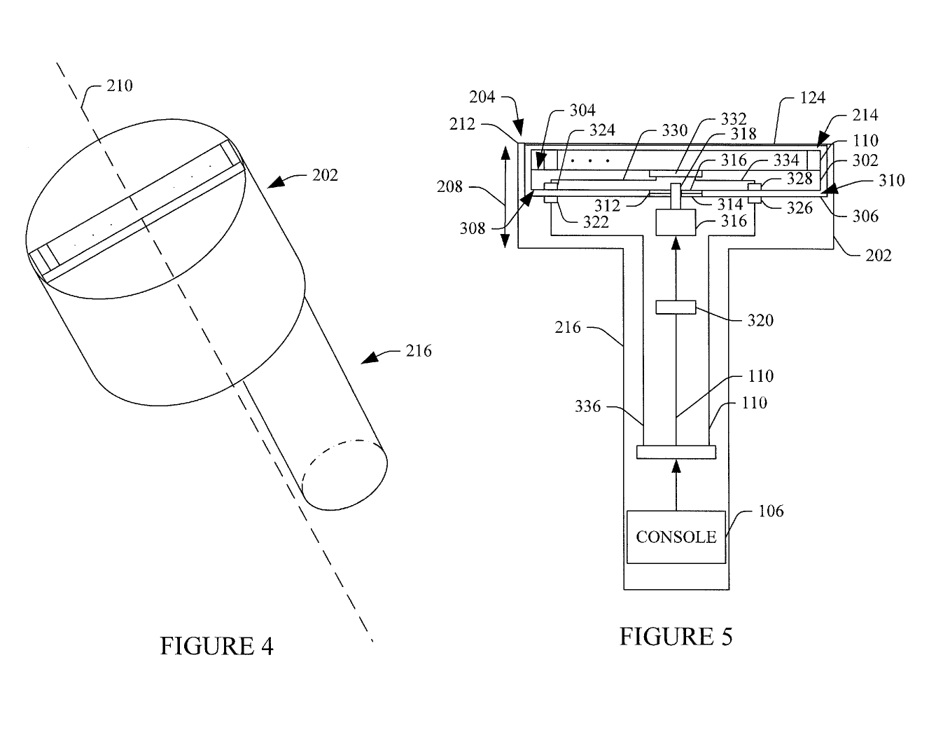

Turning to FIG. 4, a variation of the hand-held ultrasound probe 104 of FIG. 2 is illustrated. In FIG. 2, the handle portion 216 is centered along the axis 210. In FIG. 4, the handle portion 216 is not centered along the axis 210. Rather, the handle portion 216 is shifted off center relative to the axis 210. In one instance, the handle portion 216 is stationarily affixed at the off center position. In another instance, the handle portion 216 is moveably affixed to the tubular portion 202 and configured to move between at least two different positions. For example, the handle portion 216 can be moveably affixed to the tubular portion 202 to move between the centered position (FIG. 2) and one or more off center positions (e.g., FIG. 4), at least two different off center positions, etc.

Next at FIG. 5, a variation of cross sectional view of the hand-held ultrasound probe 104 of FIG. 3 is illustrated. In FIG. 3, the hand-held ultrasound probe 104 and the console 106 are different components, with the console 106 being external to the hand-held ultrasound probe 104. In FIG. 5, the console 106 is part of the hand-held ultrasound probe 104. In particular, the console 106 shown in FIG. 5 is located at least in part in the handle 216 of the hand-held ultrasound probe 104. Of course, the display 132 and the user interface 136, in this configuration, are accessible, for example, they can be integrated in the handle 216.

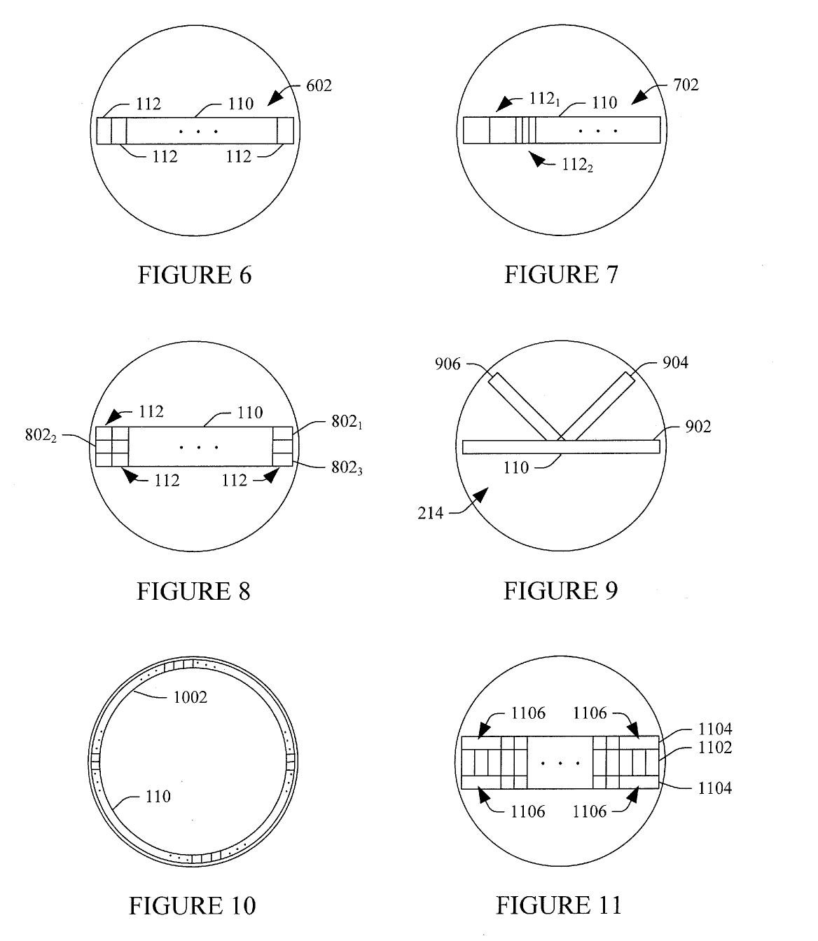

Moving to FIGS. 6, 7, 8, 9, 10 and 11, non-limiting examples of various transducer arrays 110 in connection with the embodiments disclosed herein are illustrated. Where the transducer array 110 includes a multiplicity of rows, the timing of the individual cell impulses being calculated to account for the specific rotation speed selected.

FIG. 6 shows a 1D linear array 110 with a single row 602 of equal sized transducer elements 112. FIG. 7 shows a 1D linear array 110 with a single row 702 of transducer elements 112 in which outer elements 112₁ are larger than inner elements 121₂. FIG. 8 shows an array 110 which includes three rows 802₁, 802₂, and 802₃, each with equal sized transducer elements 112. The three rows can be individual 1D arrays where only a single row of transducer elements 112 can be used at any given moment in time, or the three rows can be part of a 2D array in which all of the transducer elements 112 can be used concurrently.

FIG. 9 shows an array 110 that includes multiple 1D arrays 902, 904, and 906. The array 902 extends across the material free region 214 to about the perimeter 212, and the arrays 904 and 906 extend from a center region of the array 902 to the material free region 214 to about the perimeter 212. The arrays 904 and 906 are half as long as the array 902. FIG. 10 shows an array 110 that includes a circular array 1002 including a single circular ring of transducer elements 112. In a variation, the circular array 1002 include multiple rings. FIG. 11 shows a 1.5D or 1.75D array with a fully populated center row 1102 and sparse arrays 1104 with no elements in regions 1106.

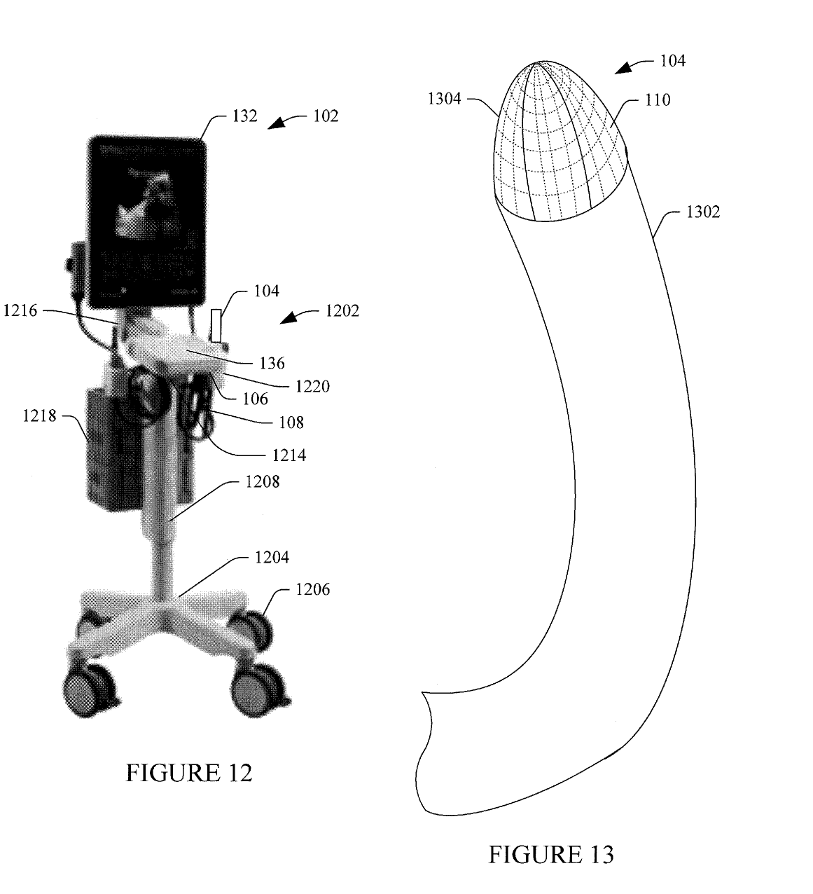

Turning to FIG. 12, an example of the ultrasound imaging system 102. In this example, the display 132 is not part of and is an external to the console 106, which is not part of and is an external to the ultrasound probe 104 is illustrated.

The console 106 and the display 132 are affixed to a transportation apparatus 1202. The transportation apparatus 1202 includes a base 1204 that include a plurality of wheels (or casters, etc.) 1206. The illustrated base 1204 includes four (4) sets of wheels 1206. However, in other embodiments, the base 1204 can include more or less sets of wheels 1206. The wheels 1206 allow the imaging system 102 to be rolled from location to location such as from examination room to examination room or other location.

The transportation apparatus 1202 further includes a post 1208 with opposing ends 1210 and 1212. The end 1210 is affixed to the base 1204, and the other end 1212 provides a first support 1214 for the console 106 and an arm support 1216 for the display 132. The post 1208 may be a fixed height or include a telescoping or otherwise height adjustable member, which can be used to adjust the height of the console 106 and the display 132.

The transportation apparatus 1202 further includes a power source 1218, which supplies power for the console 106, the display 132 and the ultrasound probe 104. The transportation apparatus 1202 further includes a probe holder 1220. In the illustrated example, the ultrasound probe 104 is located in and supported by the probe holder 1220. In the illustrated example, the path 108 includes a cable that electromechanically connects to the ultrasound probe 104 and the console 106, creating a communications path there between.

In another embodiment, the console 106 and the display 132 are affixed to a cart that does not includes wheels. In another embodiment, the display 132 is mounted to a bracket that is not affixed to the transportation apparatus 1202. For example, the display 132 is affixed to a bracket that mounts to a wall or ceiling, that rests on a desk, etc. In another example, the display 132 is part of the console 106, for instance, integrated into a side of the housing 120 and/or otherwise physically part of the console 106.

Next at FIG. 13, an embodiment in which the ultrasound probe 104 is part of and/or integrated with a catheter 1302 is illustrated. In this embodiment, the catheter 1302 includes the tubular structure 120. Furthermore, the ultrasonic window 124 includes a dome shape cover 1304 and the rotatable transducer array 110 is hemispherically shaped. A diameter of the catheter 1302 is in a range of 0.2 to 2 cm.

With this embodiment, the rotating support 302 (not visible), the transducer array 110, the stationary support 306 (not visible), the bearing 312 (not visible), the motor 316, the sensor 326 and 328, etc. can be based on micro-electromechanical systems (MEMS), nano-electromechanical systems (NEMS), and/or other technology of very small devices. Such a probe is well-suited for endo-lumen, such as endo-arterial and/or catheter tips.

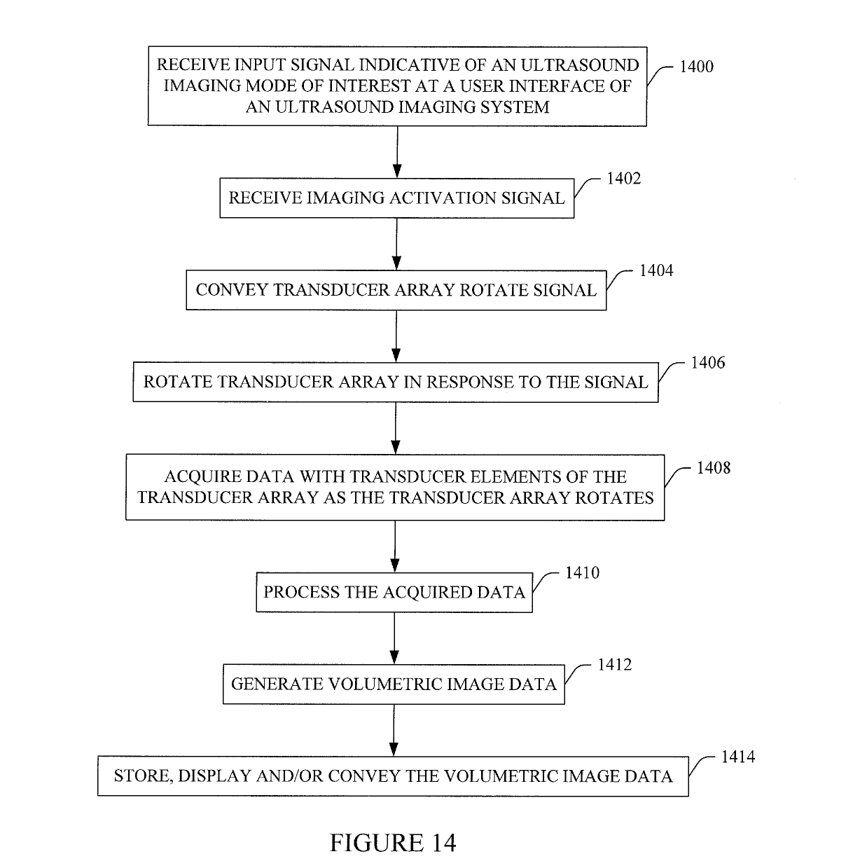

FIG. 14 illustrates a method in accordance with the description herein.

Note that the ordering of the following acts is for explanatory purposes and is not limiting. As such, one or more of the acts can be performed in a different order, including, but not limited to, concurrently. Furthermore, one or more of the acts may be omitted and/or one or more other acts may be added.

At 1400, an input signal, indicative of an ultrasound imaging mode, is received at the user interface 136 of the ultrasound probe 104. For example, in one instance, the input signal identifies a volumetric imaging mode.

At 1402, an imaging activation signal is received.

At 1404, a transducer array rotate signal is conveyed to the probe controller 320.

At 1406, the drive system 118 rotates, in response to the signal, the transducer array 110 in accordance with the identified volumetric imaging mode.

At 1408, data is acquired with the transducer elements 112 of the transducer array 110 while the transducer array 110 rotates.

At 1410, the acquired data is processed.

At 1412, volumetric image data is generated.

At 1414, the volumetric image data is at least one of stored, displayed, or conveyed to another device.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. An ultrasound imaging probe, comprising:
a housing, including a tubular portion with a first long axis and a first end region having a first non-zero diameter, a non-zero height, and an inner circular perimeter that surrounds a material free region;
a transducer array support disposed in the material free region and mechanically supported by the housing; and
a transducer array, including:
a row of transducer elements with a transducing side and a second long axis, wherein the transducer array is disposed in the material free region such that the second long axis extends within the perimeter and is perpendicular to the first long axis, and is rotatably affixed to the transducer array support on the first long axis with the transducing side facing out of the housing and the transducer array configured to rotate in a plane parallel to the circular inner perimeter.

2. The ultrasound imaging probe of claim 1, wherein the circular inner perimeter has a diameter in a range of four to twenty centimeters.

3. The ultrasound imaging probe of claim 1, further comprising:
a drive system that rotates the transducer array; and
electronics that excite the row of transducer elements to transmit ultrasound signals and receive echo signal while the transducer array rotates for a plurality of rotation angles, acquiring volumetric imaging data.

4. The ultrasound imaging probe of claim 3, further comprising:
a bearing, including: a rotating side; and a stationary side,
wherein the transducer array support includes: a rotating support affixed to the transducer array; and a stationary support affixed to the housing, wherein the rotating support and the stationary support are separated from each other by a non-zero gap; and
wherein the bearing is disposed between the rotating support and the stationary support and the rotating side affixed to the rotating support and the stationary side affixed to the stationary support.

5. The ultrasound imaging probe of claim 1, wherein the tubular portion includes a second opposing side, and the housing, further comprising: a handle portion affixed to the second opposing end of the tubular portion.

6. The ultrasound imaging probe of claim 5, wherein the handle portion has a diameter in a range of one to ten centimeters.

7. The ultrasound imaging probe of claim 5, wherein the handle portion is centered on the first long axis.

8. The ultrasound imaging probe of claim 5, wherein the handle portion is not centered on the first long axis.

9. The ultrasound imaging probe of claim 1, wherein the transducer array includes at least one of: only a single 1D row of transducer elements or a plurality of single 1D rows of transducer elements that are alternatively actuated.

10. The ultrasound imaging probe of claim 1, wherein the transducer array includes one of: a 1.5D, a 1.75D, or 2D matrix of transducer elements.

11. The ultrasound imaging probe of claim 1, wherein the transducer array includes one of: multiple rows of transducer elements orientated at different angles with respect to each other or a circular ring of transducer elements.

12. The ultrasound imaging probe of claim 1, further including: at least one of: transmit circuitry; receive circuitry; an echo processor; a display; or a user interface at least partially disposed in the housing.

13. The ultrasound imaging probe of any of claims 1 to 11, further including: a probe electro-mechanical interface to a console that includes least one of: transmit circuitry; receive circuitry; an echo processor; a display; or a user interface, wherein the console is external to the ultrasound imaging probe.

14. The ultrasound imaging probe of claim 1, wherein housing is part of a tip of an endo-lumen catheter.

15. A method, comprising:
receiving, at a user interface of a ultrasound probe, an input signal indicative of an ultrasound volumetric imaging mode;
rotating, in response to receiving the input signal, a row of transducer elements of a transducer array of an ultrasound imaging probe in an examination region plane about a central region of the a row of transducer elements;
transmitting ultrasound signals and acquiring echoes with the row of transducer elements while rotating the row of transducer elements;
processing the echoes, generating volumetric image data; and
visually presenting the generated volumetric image data.

16. The method of claim 15, wherein the row of transducer elements includes only a single 1D row of transducer elements.

17. The method of claim 15, wherein the row of transducer elements includes multiple single 1D rows of transducer elements.

18. The method of claim 15, wherein the row of transducer elements includes a circular ring of transducer elements.

19. The method of claim 15, wherein the row of transducer elements includes one of a 1.5D, a 1.75D, or a 2D array of transducer elements.

20. An ultrasound system;
an ultrasound imaging probe with a row of transducer elements;
a console that controls the row of transducer elements to transmit ultrasound signals and receive echo signals; and
means for rotating the row of transducer elements relative to the ultrasound imaging probe to acquire volumetric image data.
